(19) United States
(12) Patent Application Publication
(10) Pub. No.: US 2006/0223064 A1
(43) Pub. Date: Oct. 5, 2006

(54) Methods of nucleic acid analysis

(76) Inventor: Knut Rudi, Vestby (NO)

Correspondence Address: NEEDLE & ROSENBERG, P.C., SUITE 1000, 999 PEACHTREE STREET, ATLANTA, GA, 30309-3915, US

(21) Appl. No.: 10/561,710

(22) PCT Filed: Jun. 24, 2004
(86) PCT No.: PCT/GB04/02743

§ 371 Date: Apr. 11, 2006

(30) Foreign Application Priority Data
Jun. 27, 2003 (GB) 0315121.4

(51) Int. Cl. C12Q 1/68

(52) U.S. Cl. 435/6

(57) ABSTRACT

The present invention relates to a method of characterising a target base in a sample nucleic acid, which method comprises: (a) contacting the sample nucleic acid with an oligonucleotide primer under conditions which allow hybridisation of the oligonucleotide to the sample nucleic acid, said oligonucleotide primer being labelled with a fluorophore; (b) contacting the sample nucleic acid with a deoxynucleotide or dideoxynucleotide which is labelled with a fluorophore, under conditions which allow extension of the oligonucleotide primer through incorporation of the labelled nucleotide; and (c) measuring the fluorescence emitted by one or both of the fluorophores.

This invention relates to methods of characterising target bases in nucleic acid molecules, in particular to methods involving fluorescence detection.

Single or multiple nucleotide differences among nucleic acid molecules can be of great biological importance. The detection of such nucleotide differences has a range of applications. Major examples include the detection of point mutations for diagnostic purposes and the analysis of single nucleotide polymorphisms (SNPs) for building genetic maps of higher organisms or typing bacterial strains.

A variety of methods are currently available for the detection of SNPs (Vignal et al., 2002 Genet. Sel. Evol. Vol. 34, 275-305). These include restriction enzyme digestion, DNA strand conformation analysis, primer extension, oligonucleotide ligation assay, pyrosequencing, 5′ exonuclease detection and the invader assay. All these methods are based on the generation of an allele-specific product, followed by the detection and identification of said product. Experimental details and the drawbacks of these methods are discussed by Vignal et al.

Of these, methods based on fluorescence detection such as the 5′ exonuclease detection assay (TaqMan®) and the Invades assay are widely used because fluorescence is easy to measure. The TaqMan® assay typically involves polymerase chain reaction (PCR) amplification of the region flanking the target SNP in the presence of two probes, each probe being specific for a different allele. These probes possess a donor fluorophore at the 5′-end and an acceptor fluorophore at the 3′-end which are able to take part in a FRET (fluorescence resonance energy transfer) reaction. During the PCR, Taq polymerase unwinds and cleaves only the probe which is specific for the allele possessing the target SNP. This cleavage separates the donor fluorophore from the acceptor fluorophore, thus causing an increase in the observed fluorescence from the donor fluorophore.

The Invader® assay employs a probe with an allele-specific 3′ moiety and a 5′ flap which is added to the target nucleic acid together with an Invader® probe which anneals to said target immediately 5′ of the target SNP. Flap endonucleases (Cleavase) then cleave only the perfectly matched probe (mismatched probes can not serve as a substrate for these Flap endonucleases). This generates a fragment which in turn acts as an invader probe to mediate the Flap endonuclease cleavage of a FRET probe, thus releasing a donor fluorophore from its acceptor fluorophore and enhancing its fluorescence, typically the acceptor fluorophore acts as a quencher.

A variation of these methods is provided by fluorescence polarisation (FP), which is based on the fact that excitation of a fluorescent molecule by plane polarised light results in the emission of polarised fluorescent light from said molecule. The degree of fluorescence polarisation by a molecule, however, is proportional to the molecule's rotational relaxation time, which in turn is dependent on a range of factors such as the viscosity of the solvents, absolute temperature, gas constant and its molecular volume. Therefore, provided all other factors are held constant, fluorescence polarisation is directly proportional to the molecular volume of a molecule, which in turn is directly proportional to its molecular weight. A decrease in size (e.g. cleavage by Taq polymerase or a FLAP endonuclease) therefore results in a decrease in the FP emitted by a molecule. The advantage of FP detection is that its use can reduce the cost of the assays described above as a fluorescence quencher/acceptor is not required. However, the requirement to keep reaction conditions constant is a drawback which makes this approach less robust as any fluctuations in the reaction conditions, e.g. temperature, will interfere with the results. The use of FP in SNP genotyping is reviewed by Kwok, P. in Human Mutation (2002) 19, pp 315-323.

Recently, Xiao and Kwok (Genome Research (2003) 13(5) pp 932-939) developed an assay for SNP analysis wherein the intrinsic quenching properties of DNA are exploited (template-directed dye-terminator incorporation with fluorescence quenching detection-FQ-TD1).

Although the methods currently available are useful to qualitatively distinguish between different alleles, there is a need for a detection method which can accurately detect and quantify low levels of a target allele. For example when detecting bacterial strains in a sample, it is desirable not just to determine the presence or absence of a certain strain, but also in what proportion of the total number of bacteria the target strain is present. To our knowledge there are currently no methods available which reliably provide this quantitative information in a format which can be combined with high throughput.

A method has now been developed which addresses these problems. This method is based on the use of fluorophores as reporters in methods which characterise target bases (e.g., but not exclusively, SNPs). Instead of causing a release of a donor fluorophore from an acceptor (quencher) fluorophore, in this method a donor fluorophore is coupled to an acceptor fluorophore upon nucleotide incorporation, i.e. the base discriminating event. It represents a novel method to qualify and quantify nucleotide differences which can conveniently be carried out using standard PCR equipment in a closed tube and in real-time.

Thus, according to one aspect, the present invention provides a method of characterising a target base in a sample nucleic acid, which method comprises:
(a) contacting the sample nucleic acid with an oligonucleotide primer under conditions which allow hybridisation of the oligonucleotide to the sample nucleic acid, said oligonucleotide primer being labelled with a fluorophore;
(b) contacting the sample nucleic acid with a deoxynucleotide or dideoxynucleotide which is labelled with a fluorophore, under conditions which allow extension of the oligonucleotide primer through incorporation of the labelled nucleotide; and
(c) measuring the fluorescence emitted by one or both of the fluorophores.

Typically, a DNA sample, a fluorophore-labelled primer and deoxy- or dideoxy-nucleotides labelled with a different fluorophore are added to a standard PCR reaction mix comprising a suitable buffer and DNA polymerase. A thermo-cycling reaction is then carried out involving strand denaturation at around 95° C. for 1 min followed by a step at around 50-70° C. (depending on the nature of the target DNA and primer) during which the primers are allowed to anneal specifically to a target region of the DNA and specific incorporation of a deoxy- or dideoxy-nucleotide at the target site may occur. These steps are repeated for several cycles, e.g. between 10 and 50 cycles. During the reaction the reaction mixture is irradiated with light of a suitable wavelength and the fluorescence emitted by one or other or both of the fluorophores is recorded. Thus, step (c) will preferably comprise irradiating the sample nucleic acid (and oligonucleotide probe complex) and then measuring the fluorescence emitted by one or both of the fluorophores.

By “fluorophore” is meant a molecule (or group of atoms within a molecule) able to absorb and/or emit fluorescence. ‘Fluorescence’ is electromagnetic radiation emitted as a result of the absorption of energy derived from exciting radiation. For the purposes of the present invention the term “fluorescence” also includes phosphorescence (a type of luminescence that persists for more than about 10 ns after excitation ceases). The fluorescence emitted may for example be in the form of UV or visible light. The emission of radiation by a fluorophore is stimulated by the absorption of incident radiation and usually (i.e. except in the case of phosphorescent moiety) persists only as long as the exciting radiation is continued. Suitable sources of exciting radiation are known in the art.

By light of a “suitable wavelength” is meant light of a wavelength that is within the absorption spectrum of the donor fluorophore. Preferably, the light used for irradiation will be of a wavelength that corresponds to, or is as close as possible to, the excitation maximum of the donor fluorophore. Preferably, the wavelength is chosen to allow the best possible excitation of the donor fluorophore whilst minimizing direct excitation of the acceptor fluorophore, i.e. the wavelength falls within the absorption spectrum of the donor fluorophore but outside the absorption spectrum of the acceptor fluorophore.

If more than one donor fluorophore is used, irradiation is carried out with light of different wavelengths, wherein each wavelength is suitable for specific donor. Irradiation at different wavelengths can be carried out simultaneously or in separate steps.

According to the invention, the oligonucleotide primer is labelled with a donor fluorophore and the deoxy- or dideoxy-nucleotide is labelled with an acceptor fluorophore, or vice versa. Thus, the methods of the present invention rely on fluorescence resonance energy transfer. FRET is a process of energy transfer between two fluorophores which can occur when the emission spectrum of the first fluorophore overlaps the absorption spectrum of the second fluorophore. Quenching of the emission from the first fluorophore occurs as the excitation energy is absorbed by the second fluorophore which typically then emits its own characteristic fluorescence, i.e. fluorescence of a different wavelength. The efficiency of this energy transfer is strongly dependent on the separation of the two fluorophores. In the methods of the present invention, it has now been found that there is significant energy transfer between the two fluorophores only when the labelled primer is extended by incorporation of the labelled nucleotide. Incorporation of the labelled nucleotide is dependent on the normal rules of base-pairing and hence can be used to characterise a target base when it is known which labelled nucleotide (e.g. ddCTP or ddTTP etc.) is added to the reaction mixture.

By “donor fluorophore” is meant a reactive molecule which is capable of transferring energy to another suitably reactive molecule (acceptor fluorophore), when brought into close proximity, e.g. between 10 and 100 Å, e.g. around 60 Å or less, with said acceptor molecule. If the donor fluorophore is in isolation or located further than around 60 Å from a suitable acceptor molecule, fluorescence is freely emitted by the donor fluorophore upon irradiation.

By “acceptor fluorophore” is meant a reactive molecule which can accept radiation from a fluorophore providing the distance between the two molecules is small enough, i.e. around 60 Å or less. Thus acceptor fluorophores can absorb radiation of a suitable wavelength from other sources. The energy of the radiation absorbed by the acceptor fluorophore can either be released as fluorescence or dissipated as heat. Preferably acceptor fluorophores are used which can emit radiation as well as accept it, i.e. fluoresce.

It should be noted that the designation as “donor” and “acceptor” is not normally an intrinsic property of any particular fluorophore, but is determined by the interaction between any given pair of reactive molecules. A molecule may thus act as a donor when brought into contact with one type of fluorophore and as an acceptor when brought into contact with a different type of fluorophore. The ability of a moiety to act as donor will of course be dependent on the wavelength of the exciting radiation which is applied to the system.

The fluorescence emission maxima of the donor and the acceptor fluorophore should be sufficiently separated to allow measurement of the fluorescence emitted from either fluorophore.

Preferably, the fluorescence emission maxima of the donor and the acceptor fluorophores are as far away from one another as possible, whilst allowing overlap of the absorption spectra of the acceptor and the emission spectra of the donor. Preferably, the emission maxima of the donor and acceptor are at least 15 nm, more preferably at least 20 nm, most preferably at least 30, or 40 or 50 nm apart.

Which fluorophore is capable of transferring energy to the other fluorophore is determined by the wavelength at which each fluorophore is excited (excitation maximum): Fluorophores with lower excitation maxima are capable of transferring radiation to those with higher ones. For example, the fluorophore TAMRA (carboxytetramethylrhodamine) has an excitation maximum of around 542 nm and 6-FAM an excitation maximum of around 495 nm and consequently TAMRA acts as an acceptor and 6-FAM as a donor in this combination.

There are, however, certain reactive molecules called “dark quenchers” (Applied Biosystems, Foster City, Calif., USA) which normally dissipate any energy absorbed as heat. These represent a subclass of fluorophores which can only act as acceptors. It will be clear to the person skilled in the art which combinations of fluorophores can be used as donor and acceptor pairs. Further examples of suitable fluorophores and donor/acceptor pairs include any of 6-FAM, VIC or TET as donor fluorophores with TAMRA or MGBNFQ (Minor groove binder/non fluorescent quencher) as acceptor fluorophores, and fluorescein as a donor with Light Cycler Red 640 nm or 710 nm as an acceptor.

The methods of the present invention require labelling of the oligonucleotide primer and the nucleotide with an extrinsic fluorophore moiety. Use of any intrinsic quenching ability of nucleic acids is not envisaged.

Methods of labelling nucleotides and primers are well known in the art.

The fluorescence emitted by an acceptor fluorophore is always of a higher wavelength than the fluorescence emitted from the donor. This allows a clear distinction between the fluorescence emitted by the donor and the acceptor fluorophores and it is therefore possible to determine from any change in the fluorescence spectrum during the reaction whether and how much radiation is transferred from a donor to an acceptor. Any such radiation transfer is indicative of specific nucleotide incorporation and the amount of radiation transferred is therefore a measure of the quantity of the target allele detected.

Specific incorporation of a nucleotide at the target site can be determined by either measuring a decrease in the fluorescence emitted from the donor or by measuring an increase in the fluorescence emitted from the acceptor (or both). A background decrease in the fluorescence emitted by the donor can be expected in some cases. It may therefore be desirable to measure the increase in fluorescence from the acceptor fluorophore. Methods of recording fluorescent spectra are well known in the art. An example is the Applied Biosystems 7700 which has an integrated PCR machine.

Method of performing the necessary calculations, including normalisation, are well known in the art. The acceptor can be used for normalisation. A particularly suitable reference is a passive reference such as the ROX™ reference (Applied Biosystems). Normalisation could be done by dividing the signal measured by the signal from the reference.

The term dideoxynucleotides as used herein includes all 2′deoxynucleotides in which the 3′ hydroxyl group is absent or modified and thus, while able to be added to the primer in the presence of polymerase, is unable to enter into a subsequent polymerisation reaction. In a preferred embodiment, dideoxynucleotides or other similarly modified nucleotides are used, which cause the extension of the primer to terminate after the specific incorporation of a single nucleotide.

In another embodiment, deoxynucleotides are used. This can potentially result in the incorporation of more than one labelled or unlabelled nucleotide.

In one embodiment which utilises deoxynucleotides, energy transfer occurs from a donor on a primer to an acceptor on a nucleotide incorporated one or more bases from the end of the primer. Thus, for example, an unlabelled dATP is first incorporated then a fluorophore labelled ddTTP is incorporated resulting in FRET. Such a method could be used to characterise a doublet or the primer could anneal at a position not immediately adjacent to the target base. The method could be used, mutatis mutandis with the acceptor on the primer.

The nucleic acid molecules which can be analysed using a method according to the invention include DNA molecules which may be single or double stranded and can be of natural origin or modified or synthesized artificially. The target nucleic acid molecule may also be cDNA synthesised from RNA and the method of the invention is thus applicable to RNA analysis. The nucleic acid molecule may contain modifications and can be bound or free in solution.

The nucleic acid may be in a crude sample which includes other cellular components from the biological source from which it was obtained, e.g. a boilate of cells, but it is preferably at least partially purified. More preferably, the nucleic acid molecule is free from any or most contaminating molecules such as lipids and proteins. Methods of isolating nucleic acid from a biological sample are well known in the art. Any biological sample containing nucleic acid is a suitable source of nucleic acid and thus the sample may be derived from animals, plants, insects, bacteria, yeast, viruses or other organisms. The sample nucleic acid may be derived from one or more biological samples and may be pure or exist as part of a mixed sample.

In a preferred embodiment, a region containing the target base is amplified, e.g. by polymerase chain reaction (PCR), prior to analysis according to the invention.

In one aspect, for example, to determine whether a point mutation is present in a gene, only one species of target nucleic acid is analysed.

In another aspect, several different target nucleic acid molecules are analysed, e.g. when trying to detect the presence/absence of a certain allele in a mixed sample, e.g. when trying to determine whether a particular bacterial strain is present in a food sample.

In a further aspect, a plurality of different target sites are analysed in a single reaction, e.g. one gene is analysed for the presence of two different point mutations. The target sites may be located on a single nucleic acid molecule or be located on different nucleic acid molecules.

The term allele refers to a nucleic acid sequence which differs from another sequence for the same gene in that it contains a different base in at least one position.

Primers used according to the invention are oligonucleotides which contain sufficient sequence complementarity to a region of the target nucleic acid to be capable of sequence-specific hybridisation to said nucleic acid. The skilled person will consider what degree of sequence complementarity is required to prevent hybridisation of the primer to a non-target region and design the primer accordingly. The reaction conditions are also chosen accordingly. (By hybridisation is meant the specific pairing of complementary bases due to hydrogen bond formation). Guidance for such determinations, which are routine to the skilled man, is given for example in ‘Molecular Cloning’, a laboratory manual by Sambrook et al 1989. The primers are preferably designed such that the 3′ end of the primer hybridises immediately upstream of the target base.

In a further embodiment, the 3′ end of the primer anneals further upstream of the target base and fluorescence is transferred from the donor to the acceptor fluorophore across a plurality of unlabelled nucleotides located between the 3′ end of the primer and the target base.

The method according to the invention can be used for the characterisation of bases such as e.g. A, C, T, G, U, I or any other base which may naturally or artificially occur in a nucleic acid molecule.

The term “characterising” a target base as used herein refers either to the positive identification of a base, or to a negative identification, i.e. the confirmation that the sample nucleic acid does not contain a particular base used in the analysis at the target site. Typically the method will be performed in such a way that the exact identity of a target base is revealed, although it will be appreciated that in certain circumstances useful characterising information is obtained simply by confirming that in the sample a particular base is not found at the target position.

This characterisation is reliant upon specific incorporation of a fluorophore-labelled nucleotide (e.g. immediately) downstream of the primer through specific base-pairing with the base present in the target nucleic acid strand. The rules of base-pairing specificity are well known in the art and summarised in any standard Biology textbook.

In a preferred embodiment, the invention provides a method of characterisation of a target base in a target nucleic acid, e.g. the analysis of a point mutation in a target gene, wherein only one species of primer is used in step a) and only one species of nucleotide is used in step b).

In a further embodiment, the invention provides a method of characterisation of a target base in a target nucleic acid wherein only one species of primer is used in step a) and two or more species of nucleotide are used in step b). According to the invention, the primer may be labelled with a donor fluorophore and the nucleotides may be labelled with an acceptor fluorophore or vice versa. The different nucleotides may be labelled with the same or different fluorophores. If the purpose of the analysis is to confirm whether the target base belongs to a certain group (e.g. is a C or T), then it can be advantageous for all nucleotides to be labelled with the same fluorophore. Alternatively, each species of nucleotide could be labelled with a different fluorophore to allow a positive identification of the target base.

Thus the primer fluorophore may act as a donor to the fluorophores of all the nucleotides, regardless of whether they are different or identical. Alternatively, the primer fluorophore may act as an acceptor to the fluorophores of all nucleotides, regardless of whether they are different or identical.

It may be desirable to characterise more than one target base, e.g. two adjacent bases. For example, one might want to confirm the sequence GT at a target position. In this case, the reaction is carried out in the presence of dGTP and dTTP (or ddTTP) each labelled with a different fluorophore with such properties that the dGTP fluorophore can accept energy from the primer fluorophore and transfer it to the dTTP fluorophore, or the dTTP fluorophore can transfer energy to the dGTP fluorophore which itself transfers energy to the primer fluorophore.

Thus in one embodiment, the primer fluorophore acts as a donor to the fluorophore of one species of nucleotide which acts as an acceptor to the primer fluorophore but in turn acts as a donor to another species of nucleotide. In another embodiment, the fluorophore of one species of nucleotide acts as a donor to the fluorophore of the other species of nucleotide which acts as an acceptor to the nucleotide fluorophore but in turn acts as a donor to the primer fluorophore.

In a further embodiment, the emission from the donor is recorded (this declines as “quenching” occurs) and emission from the acceptor(s) in the higher range of the spectrum is also recorded (increases as a result of FRET). Thus it is possible to utilise a large range of the spectrum following excitation at a single wavelength.

More than one target site can be analysed in a single reaction. For example, two or more SNP sites (including single base insertions or deletions) or putative point mutations can be analysed simultaneously. The target sites may be located on a single nucleic acid or be located on different nucleic acid. For each target site, a specific primer is designed. Each primer is preferably labelled with a different fluorophore and by measuring the fluorescence emitted from the different fluorophores, base-incorporation can be analysed for each target site. Thus in another embodiment, the invention provides a method of characterisation of a base at more than one target site in one or more target nucleic acids wherein two or more primers are used in step a) of the method defined above. The same principles regarding fluorophore properties as discussed above apply. The fluorophore-labelled nucleotides may be all of the same type or there may be more than one species.

As shown in Example 3, a labelled nucleotide may act as a donor in its relationship with one labelled oligonucleotide and an acceptor with another, all in the same system.

Incorporation of a fluorophore-labelled deoxy- or dideoxynucleotide results in a measurable change in the fluorescence of the system as a whole. In order to be able to measure changes in fluorescence, electromagnetic radiation at a known wavelength is applied to the system, the wavelength being within the absorption spectrum of one of the fluorophores, the donor fluorophore. When no energy transfer takes place, the donor fluorophore will fluoresce, emitting light at a certain wavelength. The level of this emission can be considered 100% to provide a reference point for quantitative analysis. In one embodiment, referred to in the art as ‘quenching’, this signal from the donor fluorophore is measured throughout the reaction and a decrease in the signal is interpreted as incorporation of an fluorophore-labelled nucleotide resulting in energy transfer from donor to acceptor/quencher. Unless a ‘dark quencher’ is used, fluorescence by the acceptor/quencher takes place and can also be measured, although it is usually sufficient simply to measure the decrease in fluorescence by the donor.

In an alternative embodiment, the monitoring apparatus is set up to record an increase in fluorescence by the acceptor. Such a technique, where the signal that is recorded is only generated as a result of energy transfer, may be termed FRET (as opposed to quenching) although the basic principle and molecular interactions are the same in each case. A basal level will be recorded corresponding to zero nucleotide incorporation and the degree of nucleotide incorporation will be reflected in the increase in the signal measured from the acceptor fluorophore.

The methods according to the invention are simple and robust and thus suited to large-scale qualitative and/or quantitative screenings. Thus, in another embodiment, the process is automated using a robot apparatus where a large number of samples may be rapidly analysed.

The invention also provides a kit for use in a method of characterising a target base in a sample nucleic acid, which kit comprises:

a) an oligonucleotide primer labelled with a fluorophore;

b) a deoxynucleotide or dideoxynucleotide labelled with a fluorophore; and optionally

c) a polymerase

The invention will now be described in the following non-limiting Examples and with reference to the Figures in which:

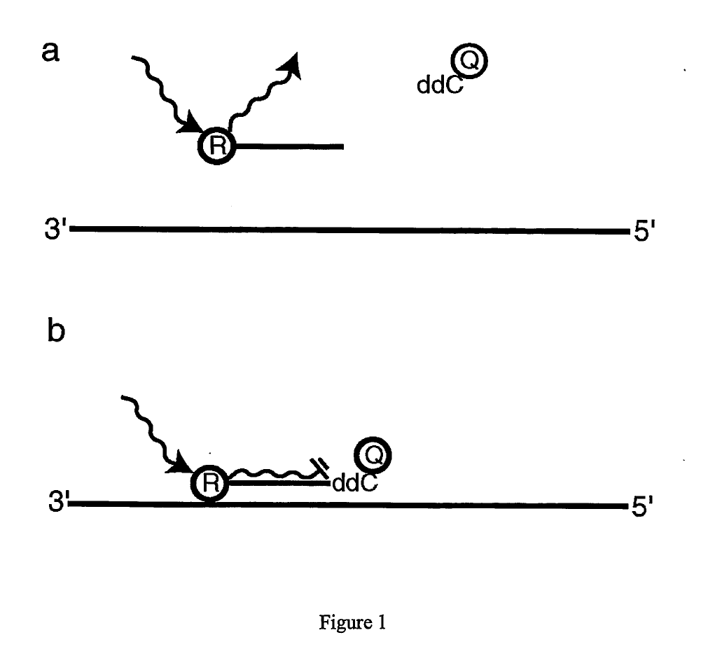

FIG. 1 is a schematic representation of one embodiment of the invention. The DNA is represented by the horizontal bars, the donor (reporter) fluorophore by a circle with R inside, and the nucleotide (in this example dideoxycytosine) labelled with the acceptor (quencher) fluorophore is represented by ddC with a circle with Q inside. (a) The situation before the incorporation of the acceptor fluorophore. The donor fluorophore emits fluorescence upon excitation. (b) The situation after the sequence-specific incorporation of the acceptor fluorophore. The fluorescence emitted from the donor fluorophore is absorbed by the acceptor fluorophore. According to this method, the emitted fluorescence is quenched and the signal measured is the reduced fluorescence from the donor/reporter; such a method is referred to herein as QEXT.

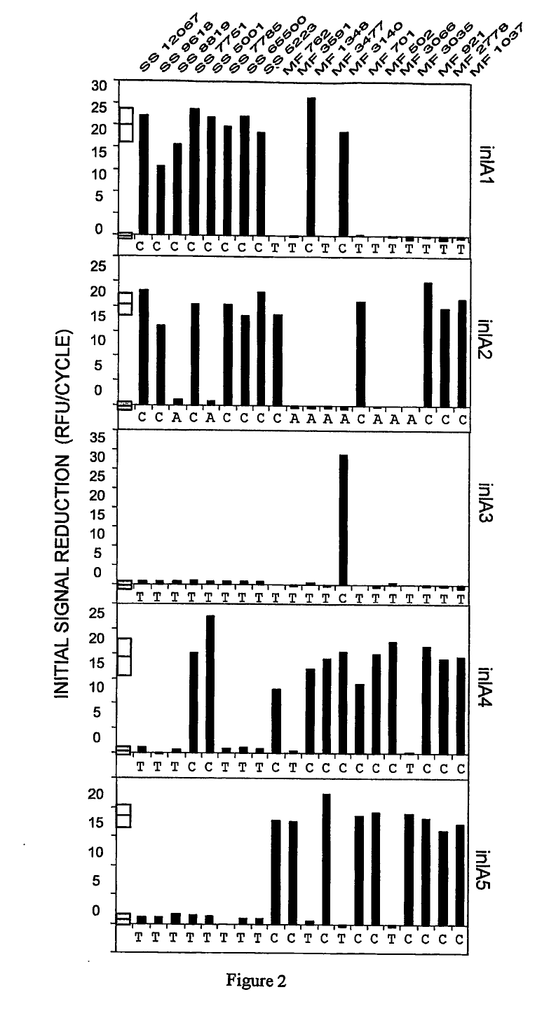

FIG. 2 is a graphical representation of the reduction in signal per cycle observed during SNP analysis of pure isolates. The QEXT method was tested on 5 known SNP sites of the inlA gene of twenty different strains of Listeria monocytogenes. For each SNP site, an oligonucleotide primer complementary to the region immediately 5′ of the SNP site was designed and labelled with the fluorescent donor fluorophore 6-FAM (inlA1-inlA5). Reactions were carried out on individual inlA PCR products from each of the 20 Listeria monocytogenes strains, using one labelled primer and dideoxycytosine (ddCTP) coupled to an acceptor fluorophore. The bars represent the initial signal reduction (indicating specific incorporation of the acceptor fluorophore).

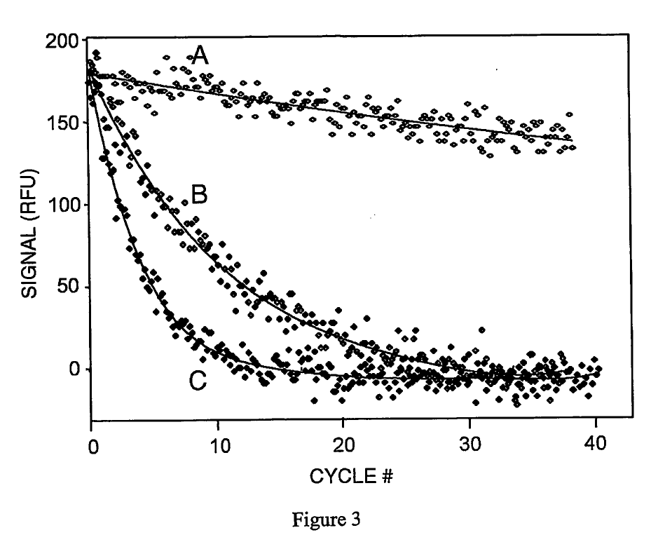

FIG. 3 is a graph showing signal reduction per cycle in a method of SNP quantification. The reduction in fluorescence measured from the donor fluorophore per cycle was used as a measure of the rate of incorporation of the acceptor fluorophore. The example shows the signal reduction for probe inlA4 for samples containing 100% target SNP (C), 25% target SNP (B), and 0% (A) (The remainder is made up of non-target SNP).

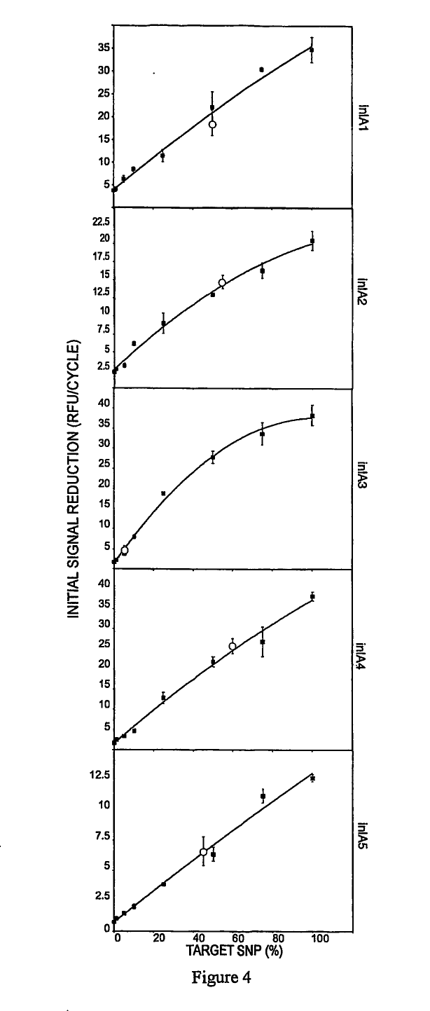

FIG. 4 is a graphical representation of signal reduction per cycle showing quantification of SNPs in mixed samples. 5 different probes containing an SNP site (inlA1 to inlA5) were analysed using PCR products of several Listeria monocytogenes strains. The allele containing the target nucleotide (CTP) at the SNP site was mixed in various proportions (0-100%) with an allele from a different strain containing a non-target nucleotide at the SNP site: inlA1-MF 3140 in MF 701, inlA2-MF 701 in MF 3140, inlA3-MF 3140 in MF 701, inlA4-MF 3140 in MF 3035, inlA5-MF 701 in MF 3140.

The initial signal reduction per PCR cycle is plotted against the percentage of the target SNP present in the sample (squares).

Target SNPs were also analysed in a background of a mixture of alleles from all 20 Listeria strains used in this study (open circles). Error bars represent standard deviations from three replicates.

### EXAMPLES

Listeria monocytogenes is a severe problem in modern food production. The fatality rate of listeriosis can be as high as 30%. However, a challenge is that this bacterium is abundant in the environment, but only a few of the subtypes are human pathogens. Tools to analyse SNPs in mixed and/or pooled samples will be of importance to understand and control L. monocytogenes.

L. monocytogenes was therefore used as a model system. The inlA gene is important for the invasion of L. monocytogenes into host cells. Five different SNP sites identified in the inlA gene were analysed. In all reactions the fluorophore 6-FAM served as a donor and the fluorophore TAMRA served as an acceptor.

### Example 1

Analysis of 5 Different SNP Sites in 20 Different Pure Isolates

A) Preparation of the Target Nucleic Acids

The strains analysed in this work are listed in Table 1.

The DNA was purified using standard procedures. The primers inlA-F (GGAGCTAACCAAATAAGTAACATCAGT-3′) and inlA-R (5′-TATCCGTACTGAAATTCCATTTAGTT-3′) were used to amplify the inlA gene from each strain. The reactions included 10 pmol primers, 200 μM of each deoxynucleotide triphosphate, 10 mM Tris-HCl (pH 8.8), 1.5 mM MgCl₂, 50 mM KCl, 0.1% Triton X-100 (wt/vol), 1 U of DynaZyme DNA polymerase (Finnzymes Oy, Espoo, Finland) and purified DNA or DNA/beads in a final volume of 50 μl. Between 30 and 35 cycles were used in the amplification reactions, with denaturation at 95° C. for 30 s, annealing for 30 s at 55° C. and extension at 72° C. for 30 s. All reactions were initiated with 4 min denaturation at 94° C. and ended with a 7 min extension at 72° C. The samples were treated with 4 units Shrimp alkaline phosphatase and 20 units exonuclease I at 37° C. for 30 min. Finally, the enzymes were inactivated at 95° C. for 10 min.

B) Characterisation of Target Bases

The 5′ 6-FAM labelled primers inlA1 (5′-CGCAGCCACTTAAGGCAATTTTTAATG-3′), inlA2 (5′-TCGGCTGGGCATAACCAAATTAGCGA-3′), inlA3 (5′GGCTTATATCACTTATATTATTAAAGTACAA-3′), inlA4 (5′-GGAAAAGGAACGACAACATTTAGTGGAAC-3′) and inlA5 (5′-AAATGCACCAGTAAACTACAAAGCAAA-3′) were all used in separate reactions. Each reaction contained 5 μl of one of the PCR products (treated as described above), 1× Thermosequenase reaction buffer, 1 pmol of one of the primers labelled with the fluorophore 6-FAM, 10 pmol of the dideoxynucleotide ddCTP labelled with the fluorophore Tamra (PerkinElmer) and 8 U of Thermosequenase (Amersham biosciences) in a total of 25 μl. The PCR analysis was done with denaturation at 95° C. for 30 s and incorporation at 55° C. for 1 min for 40 cycles. The fluorescent spectra from 500 to 660 nm were collected with an AppliedBiosystems 7700 (AppliedBiosystems, Foster City, Calif., USA) during the thermocycling. The signal reduction was recorded for each cycle during the denaturation phase by subtracting the donor signal (6-FAM) signal at 517 nm from the acceptor signal (Tamra) at 576 nm.

All the SNPs were detected with high signal to noise ratios (more than 20 fold): The reporter signal was reduced by 15 to 35 units per cycle for the target SNPs, while the non-targets gave signal reduction between −0,5 and 1.5 units (FIG. 2). The slight signal reduction for most of the negative samples was probably due to miss-incorporation by the DNA polymerase. There was also a general reduction in the total fluorescence during the thermocyling. This could be due to instability of the fluorophores.

### Example 2

Quantification of a Target Allele in a Background of Non-Target Alleles

The reaction was essentially the same as in Example 1, but instead of using a pure PCR product from a single strain, two different PCR products, each containing a different base at the target site (i.e. representing 2 different alleles), were mixed at various ratios ranging from 0:100 to 100:0. (FIGS. 3 and 4).

The initial change in the difference between the donor and the acceptor signal was used to determine the quantity of the given SNP detected (FIG. 3).

The detection limit was between 0 and 5% for all the alleles tested (the signal reduction rates for these samples were significantly different (p<0.05) as determined by a two sample t-test). Although there was a difference between the 0 and 1% samples for all five SNP's tested, these could not be separated at the p<0.05 level. In theory, the incorporation rate should be proportional to the amount of target. Linearity was obtained for all the SNP's in the range 0 to 50%-square regression coefficients (R²) of 0.99 for inlA, 0.96 for inlA2, 0.98 for inlA3, 0.99 for inlA4 and 1.0 for inlA5. The incorporation rate was lower than expected for the 75 and 100% samples for inlA 2 and 3 (see FIG. 4). This could be due to a difficulty in determining incorporation rates when there was a rapid initial decline in the reporter signal.

The frequency of the SNPs were also determined in pooled samples of the 20 strains used in this work (FIG. 4). The expected frequency for inlA1 was 50%, while the determined frequency was 41%. The corresponding values were 55 and 59% for inlA2, 5 and 5% for inlA3, 60 and 63% for inlA4 and 45 and 46% for inlA5, respectively. This shows that the QEXT assay is suitable for quantifying SNPs in pooled samples, and that the determinations can be done with an accuracy of more than 80%.

TABLE 1
L. monocytogenes strains applied in this study.

| Strain | Source^{1&2} | Origin | Serotype |
|---|---|---|---|
| 9618 | SS | unknown | 4 |
| 7751 | SS | unknown | 4 |
| 7785 | SS | unknown | 1 |
| 5001 | SS | human sporadic | 1/2a |
| 6550 | SS | unknown | 1 |
| 5223 | SS | unknown | 4 |
| 12067 | SS | human epidemic | 4b |
| 8819 | SS | human epidemic | 4b |
| 762 | MF | fish product | 1 |
| 3591 | MF | fish | 1 |
| 1348 | MF | fish processing | 1 |
| 3477 | MF | fish | 1 |
| 3140 | MF | fish product | 1 |
| 701 | MF | environment | 4 |
| 502 | MF | meat | 1 |
| 3066 | MF | fish | 1 |
| 3035 | MF | fish processing | 1 |
| 921 | MF | fish processing | 1 |
| 2778 | MF | fish processing | 1 |
| 1037 | MF | environment | 1 |
| 40 | MF | chicken | unknown |
| 39 | MF | chicken | unknown |
| 688 | MF | unknown | 4 |

¹SS: Statens Seruminstitut, Copenhagen, Denmark; MF: MATFORSK Norwegian Food Research Institute, Ås, Norway
²The SS strains are described by Rasmussen et al. (1995, Microbiology 141, 2053-2061), while the MF strains are described by Rudi et al. (2003, FEMS Microbiol Letters 220, 9-14)

### Example 3

Evaluation of Multiplex OEXT Combining Quenching and FRET Detection.

The method of the present invention was tested in a multiplex reaction using three different primers to analyse three separate target sites. Sample DNA from different Lysteria monocytogenes strains was prepared by PCR amplification and treated as described in Example 1. The primers InlA2, InlA3 and InlA4 described in Example were each labelled with a differed fluorochromes at the 5′-end. Primer InlA4 was labelled with FAM (ex 517 nm), InlA3 with TET (ex 540) and InlA2 with Texas red (ex 603 nm). The reaction was carried out in a single vessel under standard reaction conditions as described in Example 1, using Tamra (ex 577) labelled ddCTP for the incorporation.

The method was tested on 16 different bacterial strains with known SNPs at the target position. The results are shown in Table 2. The identity of the base at the target position (either C, A or T) is indicated for each SNP of each strain. The Table shows the initial change in fluorescence intensity at each wavelength measured at the first stages of the reaction.

The excitation maxima of fluorophores FAM and TET are lower than that of Tamra and fluorescence emitted from these fluorophores can therefore be quenched by Tamra. Incorporation of Tamra-labelled ddCTP at the target position adjacent to these primers is therefore indicated by a decrease in fluorescence emitted from these primer fluorophores. Conversely, incorporation of Tamra-labelled ddCTP at the target position adjacent to the TEX-labelled primer results in an increase of the fluorescence emitted from the TEX primer.

The analysis of these results is rendered a little more complicated by the fact that the emission spectra of FAM and TET overlap. The initial change in fluorescence intensity of each fluorophore must thus be interpreted in conjunction with that of the other fluorophore. Thus, in this example, if neither intensity decreases significantly, there is a T at both target positions; if the FAM intensity decreases by about twice as much as the TET intensity there is a C at the FAM target site and a T at the TET target site; and if both intensities decrease moderately there is a T at the FAM site and a C at the TET site. (C at both sites is expected to give a sharp decrease in both intensities).

This Example demonstrates that the method of the present invention is suitable for a three-plex QEXT detection where quenching and FRET detection is combined.

TABLE 2

| | inlA4 | target | | target | | target |
|---|---|---|---|---|---|---|
| Strain | FAM | base | inlA3 TET | base | InlA2 TEX | base |
| ss5223 | −1.2836 | t | −1.1089 | t | 4.2043 | c |
| ss6550 | −1.1415 | t | −0.9686 | t | 3.473 | c |
| ss7785 | 0.1475 | t | −0.1454 | t | 3.9388 | c |
| ss7751 | −10.132 | c | −6.0961 | t | 2.6058 | c |
| ss9618 | −0.6607 | t | 0.0121 | t | 2.721 | c |
| mf3591 | −1.3711 | t | −0.9562 | t | −1.1659 | a |
| mf3477 | −0.6499 | t | 0.1762 | t | −0.3242 | a |
| mf701 | −8.4022 | c | −5.1906 | t | 1.8302 | c |
| mf502 | −9.1824 | c | −4.9122 | t | −0.1404 | a |
| mf3066 | −8.0175 | c | −5.5611 | t | −1.5273 | a |
| mf3053 | −1.9645 | t | −0.9708 | t | −0.0762 | a |
| mf921 | −10.37 | c | −6.3966 | t | 2.2066 | c |
| mf1037 | −9.1868 | c | −5.7095 | t | 3.3831 | c |
| mf40 | −4.4953 | t | −5.4308 | c | 3.2317 | c |
| mf39 | −4.8287 | t | −5.8666 | c | 3.1936 | c |
| mf688 | 0.4402 | t | 0.3654 | t | 2.4298 | c |

1. A method of characterising a target base in a sample nucleic acid, which method comprises:
(a) contacting the sample nucleic acid with an oligonucleotide primer under conditions which allow hybridisation of the oligonucleotide to the sample nucleic acid, said oligonucleotide primer being labelled with a fluorophore;
(b) contacting the sample nucleic acid with a deoxynucleotide or dideoxynucleotide which is labelled with a fluorophore, under conditions which allow extension of the oligonucleotide primer through incorporation of the labelled nucleotide; and
(c) measuring the fluorescence emitted by one or both of the fluorophores.

2. A method according to claim 1, wherein one fluorophore can act as a donor and the other fluorophore can act as an acceptor.

3. A method according to claim 1 wherein the oligonucleotide primer fluorophore acts as a donor and the nucleotide fluorophore acts as an acceptor.

4. A method according to claim 1 wherein the oligonucleotide primer fluorophore acts as an acceptor and the nucleotide fluorophore acts as a donor.

5. A method according to claim 2 wherein fluorescence resonance energy transfer can take place between the donor and the acceptor fluorophore when the primer is extended by incorporation of the labelled nucleotide.

6. A method according to claim 1 wherein step b) further comprises contacting the sample with a DNA polymerase and carrying out a thermo-cycling reaction.

7. A method according to claim 1 wherein step c) comprises irradiating the sample nucleic acid and measuring the fluorescence emitted by one or both of the fluorophores.

8. A method according to claim 1 wherein the fluorescence emitted by the fluorophore of the oligonucleotide primer is recorded.

9. A method according to claim 1 wherein the fluorescence emitted by the fluorophore of the deoxynucleotide or dideoxynucleotide is recorded.

10. A method according to claim 1 wherein the primer is designed such that the 3′ end of the primer hybridises immediately upstream of the target base.

11. A method according to claim 1 wherein the labelled nucleotide is a dideoxynucleotide.

12. A method according to claim 1 wherein a plurality of target bases are characterised.

13. A method according to claim 1 wherein only one species of labelled primer is used in step a) and only one species of labelled nucleotide is used in step b).

14. A method according to claim 12 wherein one species of labelled primer and a plurality of different species of labelled nucleotides are used.

15. A method according to claim 14 wherein each species of nucleotide is labelled with a different type of fluorophore.

16. A method according to claim 12 wherein a plurality of different species of labelled primers and one species of labelled nucleotide are used.

17. A method according to claim 16 wherein each species of primer is labelled with a different type of fluorophore.

18. A method according to claim 1 wherein the fluorescence emission maxima of the two fluorophores are at least 15 nm apart.

19. A method according to claim 18 wherein the fluorescence emission maxima of the two fluorophores are at least 30 m apart.

20. A method according to claim 1 wherein the wavelength of the light used for irradiation is such that the light is only efficiently absorbed by the donor and direct excitation of the acceptor is negligible.

21. A kit for use in a method according to claim 1 which kit comprises:
a) an oligonucleotide primer labelled with a fluorophore;
b) a deoxynucleotide or dideoxynucleotide labelled with a fluorophore;
and optionally;
c) a polymerase.
